(19) United States
(12) Patent Application Publication
(10) Pub. No.: US 2020/0268835 A1
(43) Pub. Date: Aug. 27, 2020

(54) Compounds For Use in NASH

(71) Applicant: Novo Nordisk A/S, Bagsvaerd (DK)

(72) Inventors: Jesper F. Lau, Farum (DK); Thomas Kruse, Herlev (DK); Dorte Holst, Frederiksberg C (DK); Elisabeth Douglas Galsgaard, Naerum (DK); Jenny Louise Egecioglu Norlin, Ganloese (DK)

(21) Appl. No.: 16/440,591

(22) Filed: Jun. 13, 2019

(30) Foreign Application Priority Data
Feb. 27, 2019 (EP) 19159593.3

(51) Int. Cl. A61K 38/16; A61P 1/16; A61K 9/00

(52) U.S. Cl. CPC A61K 38/16; A61K 9/0019; A61P 1/16

(57) ABSTRACT

The present invention relates to compounds for use in the prevention and/or treatment of NASH. The present invention further relates to a method of preventing and/or treating NASH by administering to a subject in need thereof a compound of the invention. Compounds of the invention have been shown to be effective in reducing the relative liver weight (as percentage of total body weight), steatosis, inflammation in the liver and fibrogenesis in the liver.

The present invention relates to compounds for use in the prevention and/or treatment of non-alcoholic steatohepatitis (NASH).

### CROSS-REFERENCE TO RELATED APPLICATIONS

This application claims priority under 35 U.S.C. § 119 to European Patent Application 19159593.3, filed Feb. 27, 2019; the contents of which are incorporated herein by reference.

### INCORPORATION-BY-REFERENCE OF THE SEQUENCE LISTING

The instant application contains a Sequence Listing which has been submitted in ASCII format via EFS-Web and is hereby incorporated by reference in its entirety. Said ASCII copy, created on May 30, 2019, is named “180129US01_Sequence listing_ST25”, and is 2 kilobytes in size.

### BACKGROUND

Non-alcoholic fatty liver disease (NAFLD) is the most common chronic liver disorder in Western countries and in the USA where 30% of the adult population suffers from NAFLD. Obesity is associated with an increased risk of NAFLD, the risk increasing with increasing BMI. Furthermore, the risk rises to as much as 70-75% in patients with type 2 diabetes. Non-alcoholic fatty liver (NAFL) which is defined as simple steatosis without inflammation or fibrosis can, if not treated, progress to NASH. Approximately 20-30% of patients with simple steatosis develop NASH, which is defined by the presence of steatosis, lobular inflammation, hepatocellular ballooning degeneration with varying degrees of fibrosis that can progress into fibrous scar tissue in the liver, eventually causing cirrhosis and hepatocellular carcinoma (HCC). Approximately 10% of patients with NASH develop cirrhosis which makes NASH the third most common cause of liver transplantation in the USA and is expected to be the primary cause of liver transplantation in 2030.

Development of NASH: Simple steatosis is a prerequisite for the development of NASH and is viewed as the “first hit”. Obesity and insulin resistance play an important role in the development of hepatic steatosis. Hepatic insulin resistance favours hepatic de novo lipogenesis and insulin resistance in the adipose tissue (visceral fat in particular) will result in an increased flux of free fatty acids (FFA) from adipose tissue to the liver. The reason why a fatty liver progresses to NASH is not fully understood, but genetic as well as environmental factors may be involved. Such factors include diabetes, lipotoxicity, oxidative stress, proinflammatory cytokines, and the gut microbiome (endotoxemia), and are suggested to play a pivotal role in the development of NASH. These factors are considered to cause the “second hit”. The inflammatory microenvironment results in hepatic stellate cell activation, characterized by retinoid depletion, and fibrogenesis. Late stage hepatic fibrosis will result in widespread distortion of normal hepatic architecture and loss of liver function (cirrhosis and hepatocellular carcinoma).

Diagnosis and current treatment options: The early stages of NASH are asymptomatic. However, if it progresses to decompensated cirrhosis, in which the liver is permanently damaged, symptoms will arise. Symptoms include fatigue, unexplained weight loss, abdominal pain, swelling, bruising and bloody stools. At this point, the risk of liver failure, hepatocellular carcinoma and liver-related death is significantly increased. Furthermore, progression of fatty liver to NASH increases the risk of cardiovascular diseases and mortality. Clinically, the diagnosis of NASH requires histological confirmation by biopsy (including presence of ballooning, lobular inflammation and steatosis) together with clinical exclusion of consumption of >20 g ethanol/day. Currently, no biomarkers or scans can reliably be used as diagnostic tools and liver histology is the only way to differentiate NASH from fatty liver. A liver biopsy is considered to be the gold standard for diagnosis and treatment outcome. Due to the regenerative capacity of the liver, both NAFL and NASH are believed to be reversible if relevant treatment is initiated before scarring is too advanced. To date, no specific therapies for NAFLD or NASH exist apart from liver transplantation when reaching end stage liver disease.

### SUMMARY

In some aspects the present invention relates to a compound for use in the prevention and/or treatment of NASH, wherein said compound is selected from the group consisting of

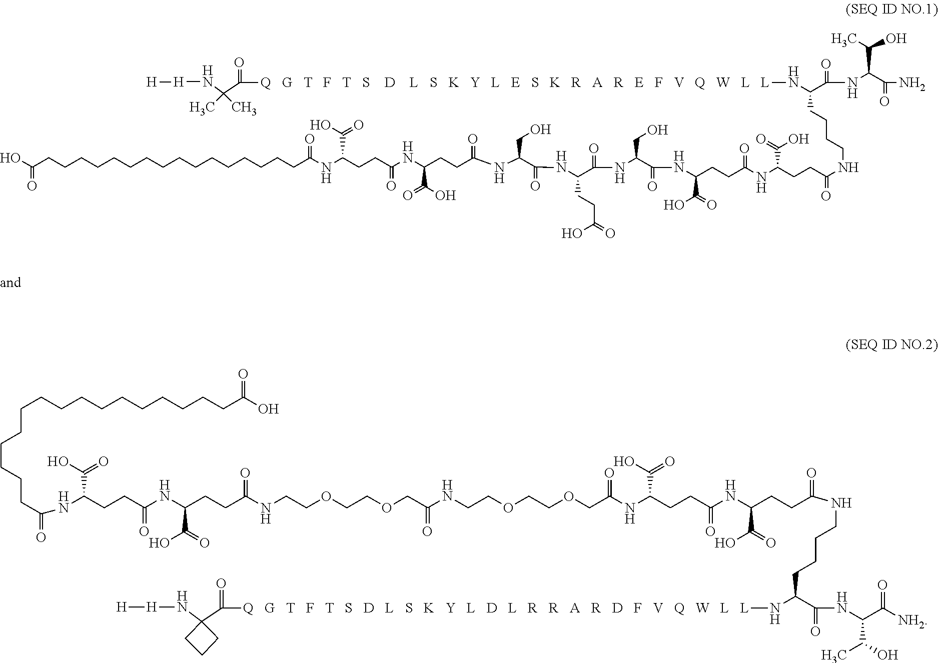

In some aspects the present invention relates to a method of preventing and/or treating NASH by administering to a subject in need thereof a compound, wherein said compound is selected from the group consisting of

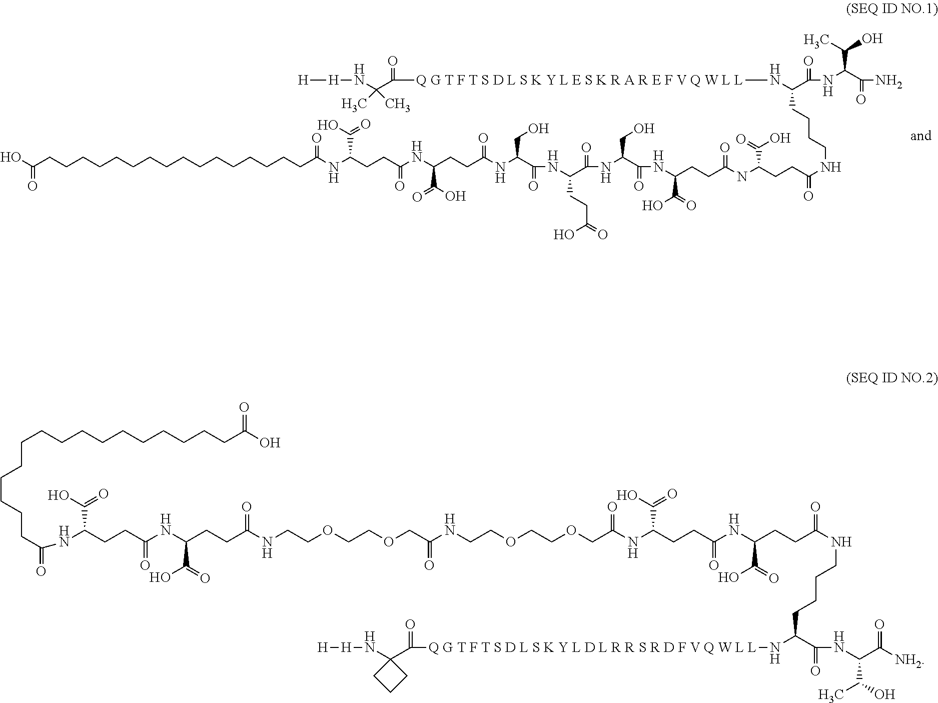

Compounds of the present invention might be useful for treating NASH. Compounds of the present invention might be useful for preventing and/or delaying NASH. Compounds of the present invention might be useful for preventing, delaying and/or reducing signs of liver damage, liver inflammation and/or liver fibrogensis.

### BRIEF DESCRIPTION OF DRAWINGS

FIG. 1 shows animal body weight in grams for day 0-56 (A) and animal body weight change in percentage for day 0-56 (B).

FIG. 2 shows relative animal liver weight as percentage of animal body weight after 8 weeks (56 days) of treatment.

FIG. 3 shows plasma alanine aminotransferase (ALT) levels after 8 weeks (56 days) of treatment.

FIG. 4 shows histopathological scoring of steatosis stage before and after 8 weeks (56 days) of treatment.

FIG. 5 shows liver triglyceride (mg/g liver) levels (A) and liver total cholesterol (mg/g liver) (B) after 8 weeks (56 days) of treatment.

FIG. 6 shows liver inflammation determined by morphometric analysis of CD45 (A) and CD11b (B) immunohistochemical staining after 8 weeks (56 days) of treatment.

FIG. 7 shows liver fibrogenesis determined by morphometric analysis of Smooth Muscle Actin (SMA) (A) and collagen III (Col III) (B) immunohistochemical staining after 8 weeks (56 days) of treatment.

FIG. 8 shows gene expression levels of collagen type I, alpha 1 (Col1A1) and collagen type III, alpha 1 (Col3A1) after 8 weeks (56 days) of treatment.

### DESCRIPTION

The present invention relates to a compound for use in the prevention and/or treatment of NASH, wherein said compound is selected from the group consisting of

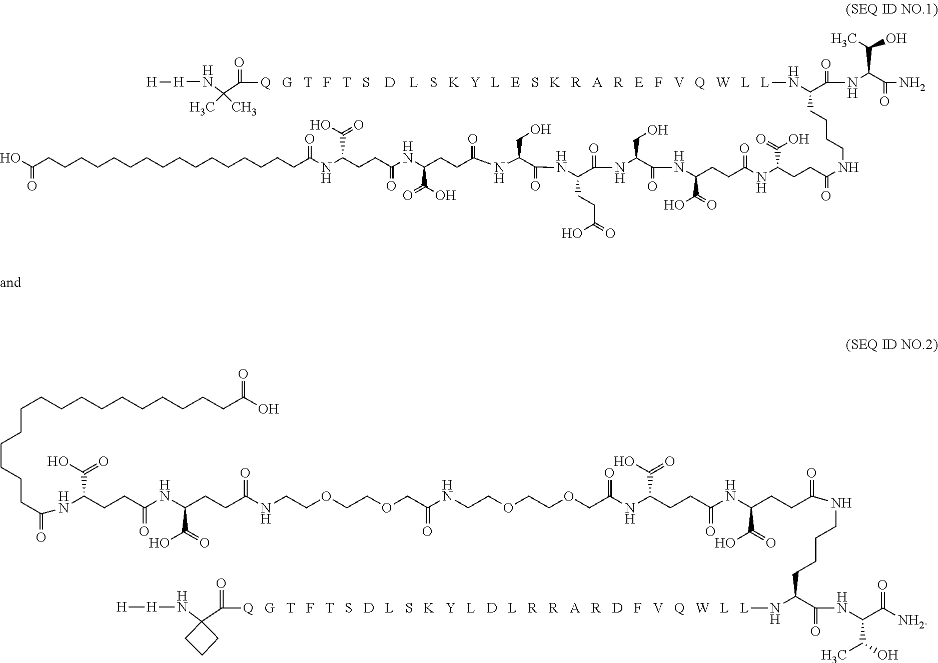

The present invention further relates to a method of preventing and/or treating NASH by administering to a subject in need thereof a compound, wherein said compound is selected from the group consisting of

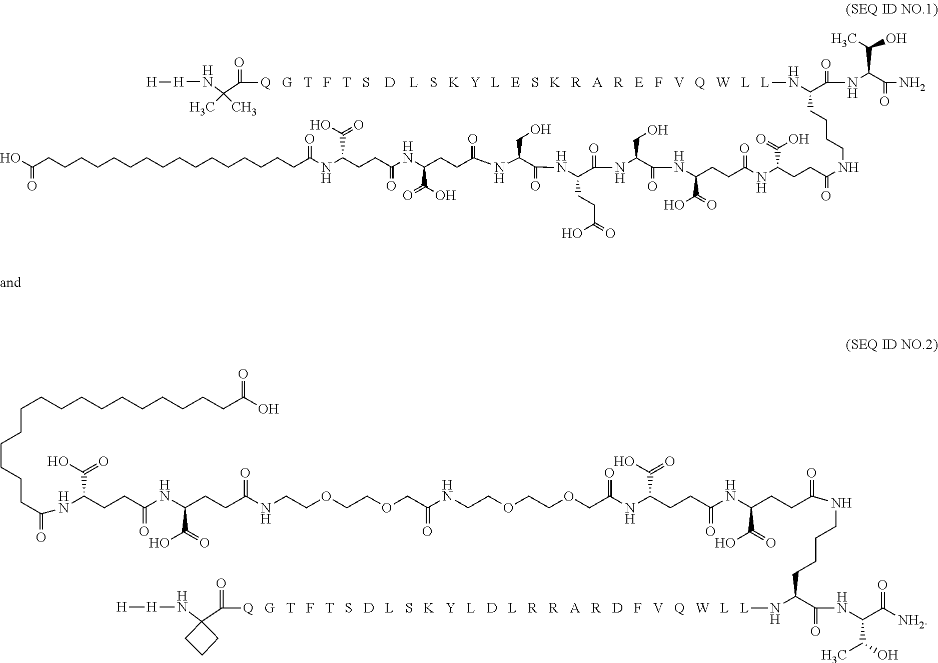

In some embodiments the present invention relates to a compound for use in the prevention and/or treatment of NASH, wherein said compound is

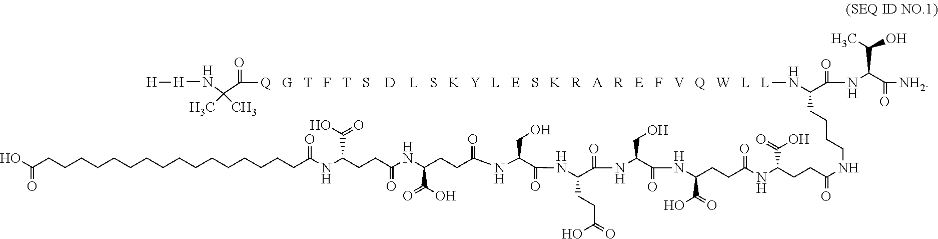

In some embodiments the present invention relates to a compound for use in the prevention and/or treatment of NASH, wherein said compound is

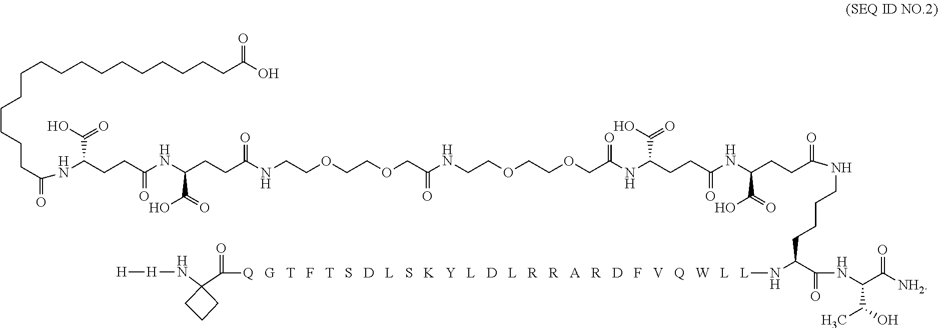

In some embodiments the present invention relates to a compound for use in the prevention and/or treatment of NASH, wherein said use prevents and/or delays increase in the relative liver weight, plasma alanine aminotransferase levels, liver triglyceride content and/or liver cholesterol. The relative liver weight is defined as liver weight as percentage of total body weight. In some embodiments said use reduces the relative liver weight, plasma alanine aminotransferase levels, liver triglyceride content and/or liver cholesterol.

In some embodiments the present invention relates to a compound for use in the prevention and/or treatment of NASH, wherein said use prevents, delays and/or reduces any histopathological signs of steatosis.

In some embodiments the present invention relates to a compound for use in the prevention and/or treatment of NASH, wherein said use prevents, delays and/or reduces inflammation in the liver.

In some embodiments the present invention relates to a compound for use in the prevention and/or treatment of NASH, wherein said use prevents, delays and/or reduces fibrogenesis in the liver.

In some embodiments the present invention relates to a compound for use in the prevention and/or treatment of NASH, wherein said compound is administered in the form of a pharmaceutical composition comprising 1-10 mg/ml compound.

In some embodiments the present invention relates to a compound for use in the prevention and/or treatment of NASH, wherein the dosage of said compound is in the range from 0.01 to 10 mg.

In some embodiments the present invention relates to a compound for use in the prevention and/or treatment of NASH, wherein said compound is subcutaneously administered once weekly. In some embodiments said compound is administered for at least 12 months.

In some embodiments the present invention relates to a compound for use in the prevention and/or treatment of NASH, wherein said compound is administered in a therapeutically effective amount to a subject in need thereof. In some embodiments said subject is obese and/or has diabetes. In some embodiments said subject suffers from overweight, obesity, hyperglycemia, type 2 diabetes, impaired glucose tolerance and/or type 1 diabetes.

BMI (body mass index) is a measure of body fat based on height and weight. The formula for calculation is BMI=(weight in kilograms)/(height in meters)². In some embodiments the present invention relates to a compound use in the prevention and/or treatment of NASH, wherein said compound is administered in a therapeutically effective amount to a subject in need thereof, wherein said subject has a BMI of at least 25 kg/m². In some embodiments said subject has a BMI of at least 30 kg/m². In some embodiments said subject has a BMI between 30-50 kg/m².

In some embodiments the present invention relates to a method of preventing and/or treating NASH by administering to a subject in need thereof a compound, wherein said compound is

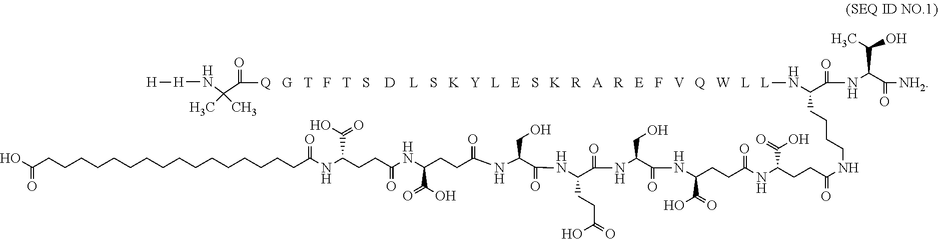

In some embodiments the present invention relates to a method of preventing and/or treating NASH by administering to a subject in need thereof a compound, wherein said compound is

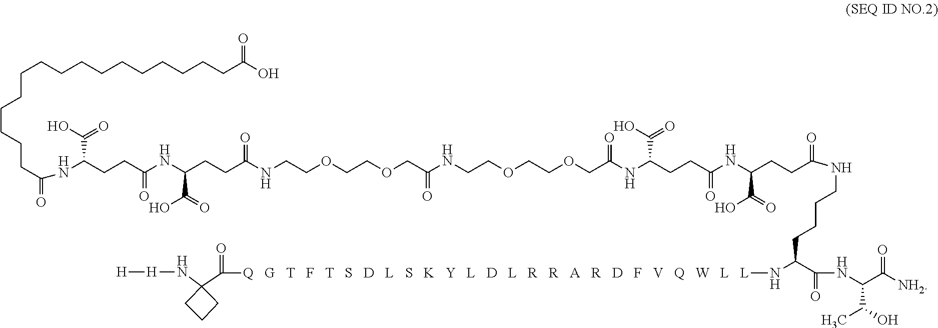

In some embodiments the present invention relates to a method of preventing and/or treating NASH by administering to a subject in need thereof a compound, wherein said method prevents and/or delays increase in the relative liver weight, plasma alanine aminotransferase levels, liver triglyceride content and/or liver cholesterol. In some embodiments said method reduces the relative liver weight, plasma alanine aminotransferase levels, liver triglyceride content and/or liver cholesterol.

In some embodiments the present invention relates to a method of preventing and/or treating NASH by administering to a subject in need thereof a compound, wherein said method prevents, delays and/or reduces any histopathological signs of steatosis.

In some embodiments the present invention relates to a method of preventing and/or treating NASH by administering to a subject in need thereof a compound, wherein said method prevents, delays and/or reduces inflammation in the liver.

In some embodiments the present invention relates to a method of preventing and/or treating NASH by administering to a subject in need thereof a compound, wherein said method prevents, delays and/or reduces fibrogenesis in the liver.

In some embodiments the present invention relates to a method of preventing and/or treating NASH by administering to a subject in need thereof a compound, wherein said compound is administered in the form of a pharmaceutical composition comprising 1-10 mg/ml compound.

In some embodiments the present invention relates to a method of preventing and/or treating NASH by administering to a subject in need thereof a compound, wherein the dosage of said compound is in the range from 0.01 to 10 mg.

In some embodiments the present invention relates to a method of preventing and/or treating NASH by administering to a subject in need thereof a compound, wherein said compound is subcutaneously administered once weekly. In some embodiments said compound is administered for at least 12 months.

In some embodiments the present invention relates to a method of preventing and/or treating NASH by administering to a subject in need thereof a compound, wherein said compound is administered in a therapeutically effective amount to a subject in need thereof.

In some embodiments the present invention relates to a method of preventing and/or treating NASH by administering to a subject in need thereof a compound, wherein said subject is obese and/or has diabetes. In some embodiments said subject suffers from overweight, obesity, hyperglycemia, type 2 diabetes, impaired glucose tolerance and/or type 1 diabetes.

In some embodiments the present invention relates to a method of preventing and/or treating NASH by administering to a subject in need thereof a compound, wherein said subject has a BMI of at least 25 kg/m². In some embodiments said subject has a BMI of at least 30 kg/m². In some embodiments said subject has a BMI between 30-50 kg/m².

### Subject and Subpopulations

The subject to be administered a compound according to the present invention may be human, such as an adult human. In some embodiments said subjects are adults.

In some embodiments the subject to be administered a compound according to the present invention suffers from overweight, obesity, hyperglycemia, type 2 diabetes, impaired glucose tolerance and/or type 1 diabetes and/or an BMI of at least 25 kg/m², alternatively at least 30 kg/m², alternatively between 30-50 kg/m².

### Compounds of the Invention

In one embodiment of the present invention the compound is N^{ε28}-[(4S)-4-carboxy-4-[[(4S)-4-carboxy-4-[[(2S)-2-[[(2S)-4-carboxy-2-[[(2S)-2-[[(4S)-4-carboxy-4-[[(4S)-4-carboxy-4-(17-carboxyheptadecanoylamino)butanoyl]amino]butanoyl] amino]-3-hydroxypropanoyl]amino]butanoyl]amino]-3-hydroxypropanoyl]amino]butanoyl] amino]butanoyl]-[Aib2,Leu10,Glu15,Lys17,Arg20,Glu21,Leu27,Lys28]-Glucagon amide. The compound comprises the amino acid sequence given as SEQ ID NO: 1 and is a C-terminal amide. This compound may be prepared as described in Example 54 of WO2014/170496.

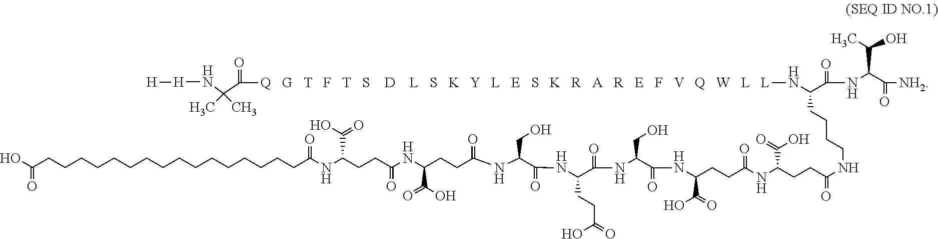

In one embodiment of the present invention the compound is N^{ε28}-[(4S)-4-carboxy-4-[[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy] ethoxy]acetyl]amino]ethoxy]ethoxy]acetyl]amino]butanoyl]amino]butanoyl]-[Acb2,Leu10,Leu16,Arg20,Leu27,Lys28]-Glucagon amide. The compound comprises the amino acid sequence given as SEQ ID NO: 2 and is a C-terminal amide. This compound may be prepared as described in Example 45 of WO2014/170496.

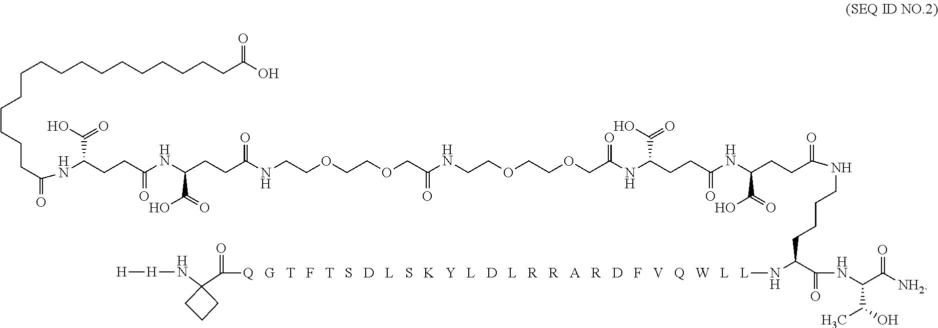

### Receptor Binding and Activation

A receptor agonist may be defined as a peptide that binds to a receptor and elicits a response typical of the natural ligand. Thus, for example, a “GLP-1 receptor agonist” may be defined as a compound which is capable of binding to the GLP-1 receptor and capable of fully or partially activating it. The term “glucagon receptor agonist” as used herein refers to any compound which is capable of binding to the glucagon receptor and capable of fully or partially activating it. A GLP-1/glucagon receptor co-agonist may be defined as a peptide that is able to activate both the GLP-1 and the glucagon receptors. The term “GLP-1 activity” refers to the ability to bind to the GLP-1 receptor and initiate a signal transduction pathway resulting in insulinotropic action or other physiological effects as is known in the art.

In some embodiments of the present invention, the compounds are GLP-1 receptor agonists. In some embodiments, the compounds are glucagon receptor agonists. In some embodiments the compounds are GLP-1/glucagon receptor co-agonists. In some embodiments the compounds have GLP-1 activity.

### Biological Activity—In Vitro Affinity and Potency

In one embodiment affinity refers to in vitro binding affinity, i.e. performance in a GLP-1 receptor binding affinity assay and in a glucagon receptor binding affinity assay, more particularly to the capability of binding the human GLP-1 receptor and to the human glucagon receptor. The binding affinity of the human GLP-1 receptor may be measured in a binding assay, e.g. in a stably transfected BHK cell line that expresses the human GLP-1 receptor. Radioactively labelled GLP-1 binds to the receptor and may be displaced competitvely by a compound. Binding of radioligand may be determined in the presence of scintillation proximity assay (SPA) beads which bind to cell membranes and when radioactivity is close to the bead it produces light which is measured and is a measure of the in vitro binding affinity. One non-limiting example of such an assay is described in Example 1 herein. The binding affinity of the human glucagon receptor may be measured in a binding affinity assay, e.g. in a stably transfected BHK cell line that expresses the human glucagon receptor. Radioactively-labelled glucagon binds to the receptor and may be displaced competively by a compound. Binding of radioligand may be determined in the presence of scintillation proximity assay (SPA) beads which bind to cell membranes and when radioactivity is close to the bead it produces light which is measured and is a measure of the in vitro binding affinity.

The term half maximal inhibitory concentration (IC₅₀) generally refers to the concentration of competing compound which displaces 50% of the specific binding of the radioligand binding corresponding to halfway between the baseline and maximum, by reference to the dose response curve. IC₅₀ is used as a measure of the binding affinity of a compound and represents the concentration where 50% of its maximal binding is observed.

The in vitro binding of the compounds of the invention may be determined as described above, and the IC₅₀ values of the compounds are determined. The lower the IC₅₀ value, the better the binding affinity.

The affinity, i.e. IC₅₀, of the compounds on the GLP-1 receptor and glucagon receptor may be determined by the assay described in Example 1 herein.

In some embodiments of the present invention, the compounds have an in vitro binding affinity on the GLP-1 receptor determined using the method of Example 1 herein corresponding to an IC₅₀ at or below 100 nM, alternatively below 10 nM, alternatively below 5 nM, alternatively below 3 nM.

In some embodiments of the present invention, the compounds have an in vitro binding affinity on the glucagon receptor determined using the method of Example 1 herein corresponding to an IC₅₀ at or below 100 nM, alternatively below 50 nM, alternatively below 10 nM, preferable below 5 nM, alternatively below 3 nM.

In some embodiments of the present invention, the compounds have an in vitro binding affinity on the GLP-1 receptor and the glucagon receptor determined using the method of Example 1 herein corresponding to an IC₅₀ at or below 100 nM, alternatively below 10 nM, alternatively below 5 nM, alternatively below 3 nM.

### Pharmaceutical Composition

A compound of the invention may be administered in the form of a pharmaceutical composition. The pharmaceutical composition may comprise a compound of the invention in a concentration from 0.01 mg/ml to 100 mg/ml. In some embodiments the pharmaceutical composition comprises 0.01-50 mg/ml, or 0.01-20 mg/ml, or 0.01-10 mg/ml compound of the invention. In some embodiments the pharmaceutical composition comprises 0.1-20 mg/ml compound of the invention.

The pharmaceutical compositions described herein may further comprise one or more pharmaceutically acceptable excipients, for example selected from the group consisting of buffer system, preservative, tonicity agent, chelating agent, stabilizer and surfactant. In some embodiments the pharmaceutical composition comprises one or more pharmaceutically acceptable excipients, such as one or more selected from the group consisting of a buffer, an isotonic agent, and a preservative. The formulation of pharmaceutically active ingredients with various excipients is known in the art, see e.g. Remington: The Science and Practice of Pharmacy (e.g. 19th edition (1995), and any later editions). The term “excipient” broadly refers to any component other than the active therapeutic ingredient(s), e.g. compounds of the invention. The excipient may be an inert substance, an inactive substance, and/or a not medicinally active substance.

In some embodiments the pharmaceutical composition comprises a phosphate buffer, such as a sodium phosphate buffer, e.g. disodium phosphate. In some embodiments the pharmaceutical composition comprises an isotonic agent, such as propylene glycol. In some embodiments the pharmaceutical composition comprises a preservative, such as phenol.

The pharmaceutical composition may be in the form of a solution or a suspension. In some embodiments the pharmaceutical composition is aqueous composition, such as an aqueous solution or an aqueous suspension. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water. An aqueous composition may comprise at least 50% w/w water, or at least 60%, 70%, 80%, or even at least 90% w/w of water. In some embodiments the pharmaceutical composition has a pH in the range of 7.0-9.0, such as 7.0-8.5.

### Administration Regimen

A compound of the invention may be administered in a therapeutically effective amount, such as an amount therapeutically effective to prevent and/or treat NASH. The therapeutically effective amount of compounds of the invention can be assessed by a medical doctor. The dosage of compounds of the invention may be in the range from 0.01 to 10 mg, alternatively from 0.5 to 7 mg, alternatively from 2 to 6 mg.

A compound of the invention may be administered once weekly or more frequent, such as once daily. In some embodiments a compound of the invention is administered at any time in the day. In some embodiments a compound of the invention is administered subcutaneously. In some embodiments the dosage of a compound of the invention is in the range from 0.5 to 7.0 mg, such as in the range from 2.0 to 6.0 mg. In some embodiments the weekly dosage of a compound of the invention is selected from the group consisting of 0.5, 1, 2, 3, 4, 5, 6 and 7 mg.

In some embodiments the term “chronic treatment” as used herein with reference to a compound of the invention means administration in an amount and frequency to provide a therapeutic effect. In some embodiments the term “chronic treatment” as used herein with reference to a compound of the invention means administration of a compound for a long period of time, such as at least one year, alternatively at least five years.

Unless otherwise stated, ranges herein include their end points. In some embodiments the term “a” means “one or more”. In some embodiments, and unless otherwise indicated in the specification, terms presented in singular form also include the plural situation. Herein the term “about” means±10% of the value referred to, and includes the value.

### Non-Limiting Embodiments of the Invention

Non-limiting embodiments of the invention include:
1. A compound for use in the prevention and/or treatment of NASH, wherein said compound is selected from the group consisting of

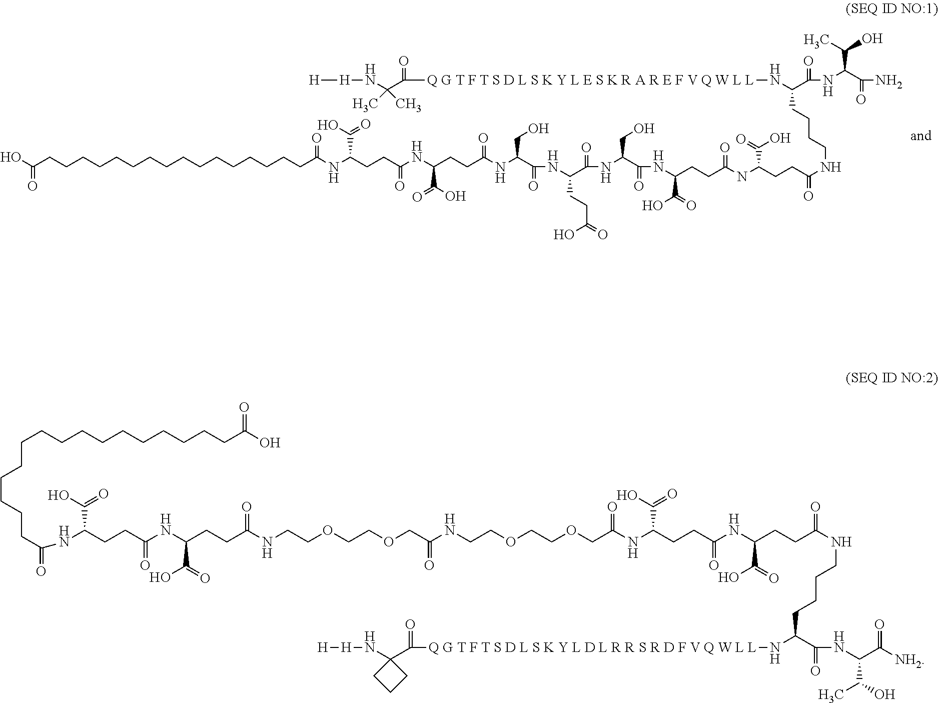

2. A compound use in the prevention and/or treatment of NASH, wherein said compound is

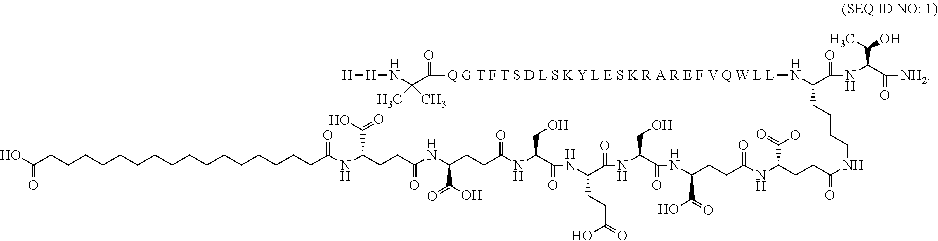

3. A compound use in the prevention and/or treatment of NASH, wherein said compound is

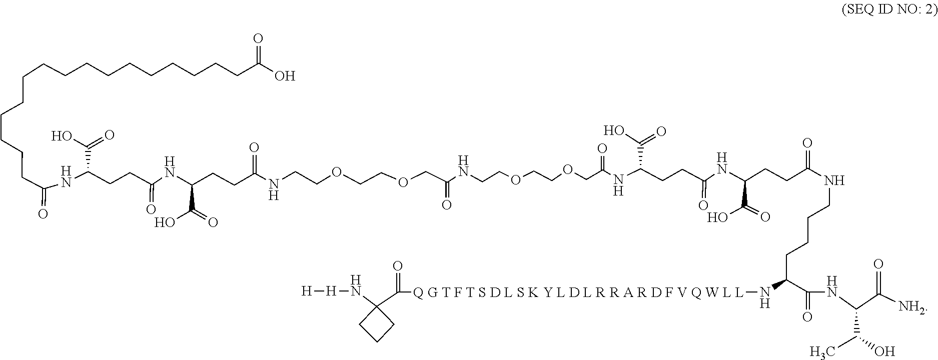

4. The compound for use according to any of the preceding embodiments, wherein said use prevents and/or delays increase in the relative liver weight, plasma alanine aminotransferase levels, liver triglyceride content and/or liver cholesterol.
5. The compound for use according to any of the preceding embodiments, wherein said use reduces the relative liver weight, plasma alanine aminotransferase levels, liver triglyceride content and/or liver cholesterol.
6. The compound for use according to any of the preceding embodiments, wherein said use prevents, delays and/or reduces any histopathological signs of steatosis.
7. The compound for use according to any of the preceding embodiments, wherein said use prevents, delays and/or reduces inflammation in the liver.
8. The compound for use according to any of the preceding embodiments, wherein said use prevents, delays and/or reduces fibrogenesis in the liver.
9. The compound for use according to any of the preceding embodiments, wherein said compound is a GLP-1/glucagon receptor co-agonist.
10. The compound for use according to any of the preceding embodiments, wherein said compound is administered in the form of a pharmaceutical composition comprising 1-10 mg/ml compound.
11. The compound for use according to any of the preceding embodiments, wherein the dosage of said compound is in the range from 0.01 to 10 mg.
12. The compound for use according to any of the preceding embodiments, wherein said compound is subcutaneously administered once weekly.
13. The compound for use according to any of the preceding embodiments, wherein said compound is administered for at least 12 months.
14. The compound for use according to any of the preceding embodiments, wherein said compound is administered in a therapeutically effective amount to a subject in need thereof.
15. The compound for use according to embodiment 14, wherein said subject is obese and/or has diabetes.
16. The compound for use according to embodiment 14, wherein said subject suffers from overweight, obesity, hyperglycemia, type 2 diabetes, impaired glucose tolerance and/or type 1 diabetes.
17. The compound for use according to any one of embodiments 14-16, wherein said subject has a BMI of at least 25 kg/m².
18. The compound for use according to any one of embodiments 14-16, wherein said subject has a BMI of at least 30 kg/m².
19. The compound for use according to any one of embodiments 14-16, wherein said subject has a BMI between 30-50 kg/m².
20. A pharmaceutical composition for use in the prevention and/or treatment of NASH, wherein said pharmaceutical composition comprises a compound selected from the group consisting of

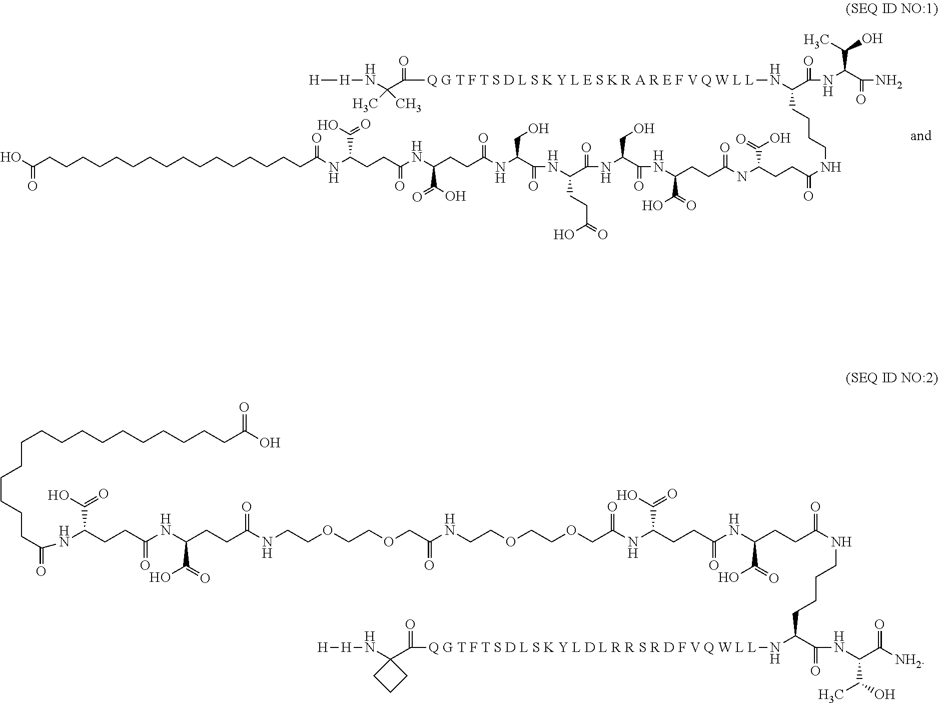

21. The pharmaceutical composition for use according to embodiment 20, wherein said pharmaceutical composition comprises 1-10 mg/ml of said compound.
22. The pharmaceutical composition for use according to any of embodiments 20 or 21, wherein said pharmaceutical composition further comprises one or more pharmaceutically acceptable excipients.
23. A method of preventing and/or treating NASH by administering to a subject in need thereof a compound, wherein said compound is selected from the group consisting of

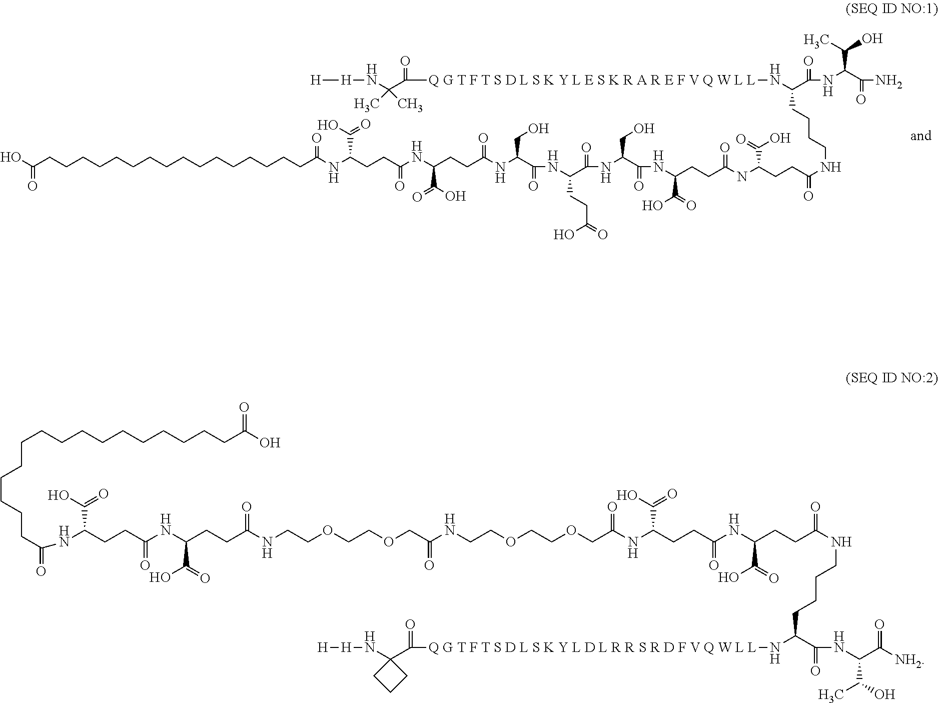

24. A method of preventing and/or treating NASH by administering to a subject in need thereof a compound, wherein said compound is

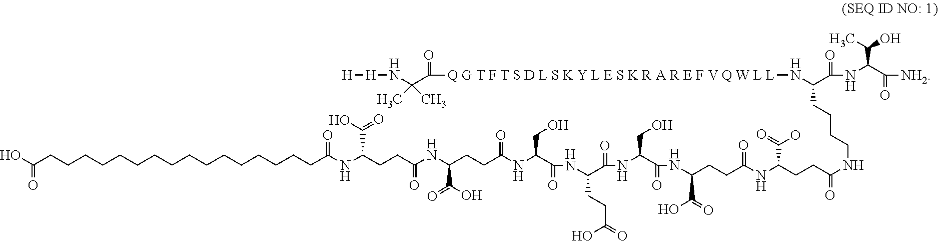

25. A method of preventing and/or treating NASH by administering to a subject in need thereof a compound, wherein said compound is

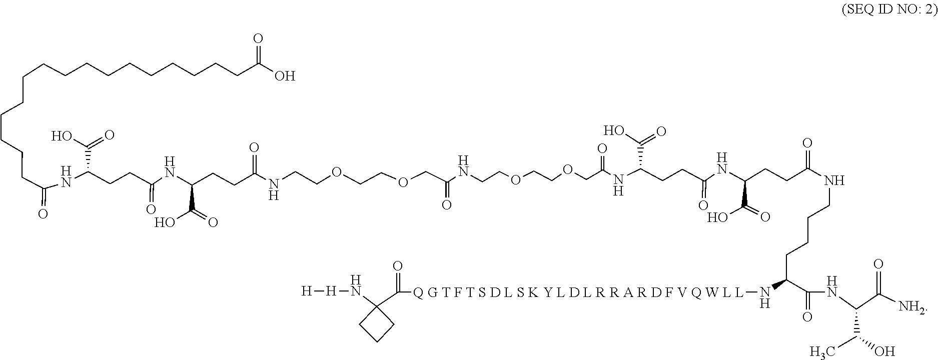

26. The method according to any of embodiments 23-25, wherein said method prevents and/or delays increase in the relative liver weight, plasma alanine aminotransferase levels, liver triglyceride content and/or liver cholesterol.
27. The method according to any of embodiments 23-26, wherein said method reduces the relative liver weight, plasma alanine aminotransferase levels, liver triglyceride content and/or liver cholesterol.
28. The method according to any of embodiments 23-27, wherein said method prevents, delays and/or reduces any histopathological signs of steatosis.
29. The method according to any of embodiments 23-28, wherein said method prevents, delays and/or reduces inflammation in the liver.
30. The method according to any of embodiments 23-29, wherein said method prevents, delays and/or reduces fibrogenesis in the liver.
31. The method according to any of embodiments 23-30, wherein said compound a GLP-1/glucagon receptor co-agonist.
32. The method according to any of embodiments 23-31, wherein said compound is administered in the form of a pharmaceutical composition comprising 1-10 mg/ml compound.
33. The method according to any of embodiments 23-32, wherein the dosage of said compound is in the range from 0.01 to 10 mg.
34. The method according to any of embodiments 23-33, wherein said compound is subcutaneously administered once weekly.
35. The method according to any of embodiments 23-34, wherein said compound is administered for at least 12 months.
36. The method according to any of embodiments 23-35, wherein said compound is administered in a therapeutically effective amount to a subject in need thereof.
37. The method according to any of embodiments 23-36, wherein said subject is obese and/or has diabetes.
38. The method according to any of embodiments 23-37, wherein said subject suffers from overweight, obesity, hyperglycemia, type 2 diabetes, impaired glucose tolerance and/or type 1 diabetes.
39. The method according to any of embodiments 23-38, wherein said subject has a BMI of at least 25 kg/m².
40. The method according to any of embodiments 23-38, wherein said subject has a BMI of at least 30 kg/m².
41. The method according to any of embodiments 23-38, wherein said subject has a BMI between 30-50 kg/m².
42. A method of preventing and/or treating NASH by administering to a subject in need thereof a pharmaceutical composition, wherein said pharmaceutical composition comprises a compound selected from the group consisting of

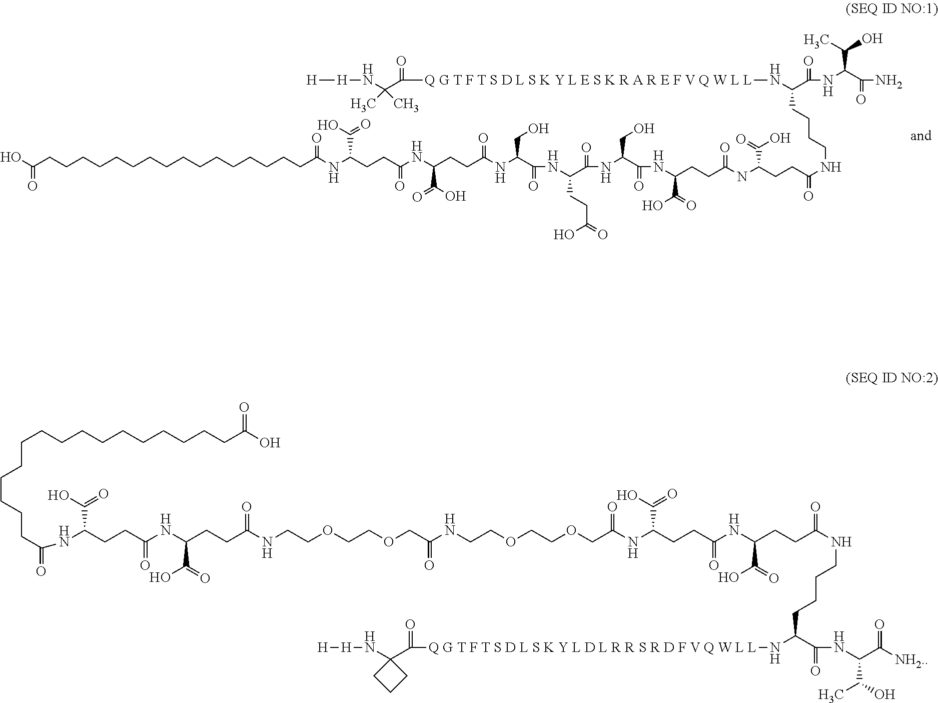

43. The method according to embodiment 42, wherein said pharmaceutical composition comprises 1-10 mg/ml of said compound.
44. The method according to any of embodiments 42 or 43, wherein said pharmaceutical composition further comprises one or more pharmaceutically acceptable excipients.

### EXAMPLES

### List of Abbreviations

ALT: Alanine transferase
ANOVA: Analysis of variance
BHK: Baby hamster kidney
BW: Body weight
CD11 b: Cluster of Differentiation 11 b.
CD45: Cluster of Differentiation 45.
Col: Collagen
Col III: Collagen III
Col1A1: Collagen type I, alpha 1
Col3A1: Collagen type III, alpha 1
HE: Hematoxylin and Eosin
IC₅₀: Half maximal inhibitory concentration
NASH: Non-alcoholic steatohepatitis
PFA: Paraformaldehyde
RNA: Ribonucleic acid
ROI: Region of interest
SC: Subcutaneously
SEM: Standard error of mean
SMA: Smooth Muscle Actin
SPA: Scintillation proximity assay
SR: Sirius Red
STAR: Spliced Transcripts Alignment to a Reference
VIS: Visiopharm Integrator System
WGA: Wheat germ agglutinin
WM: Weight-matched

### General Methods of Animal Experiments

### Animals, Housing and Diet

All animal experiments were conducted in accordance with Gubra bioethical guidelines, which are fully compliant to internationally accepted principles for the care and use of laboratory animals. The described experiments were covered by personal licenses for Jacob Jelsing (2013-15-2934-00784) issued by the Danish Committee for animal research.

At 5 weeks of age, male C57BL/6 mice were purchased from JanVier, France. During the acclimatization and diet-induction period, the mice were group housed ten per cage in custom-made cabinets under a 12:12 light dark cycle (lights on from 3 AM-3 PM) at controlled temperature conditions (22±1° C.; 50±10% relative humidity). Throughout the diet-induction the mice had ad libitum access to AMLN diet (D09100301, Research Diet, USA) (40% fat (18% trans-fat), 40% carbohydrates (20% fructose) and 2% cholesterol) or regular rodent chow (chow vehicle group) (Altromin 1324, Brogaarden, Denmark) and tap water. Throughout the study period the mice had ad libitum access to AMLN diet, except the weight-matched group which was food restricted to lower the body weight to the same level as for animals receiving compound A or compound B, or regular rodent chow (chow vehicle group) and tap water. The animals were kept on the diet for 34 weeks before pre-biopsy and maintained on diet throughout the study period. During post-operative recovery and throughout the study period, all animals were single-housed.

Allocation into Studies, Stratification, Randomization and Baseline Monitoring

During the diet-induction period, body weight was intermittently monitored. After 34 weeks of diet-induction (three weeks prior to study start), a liver biopsy was obtained for hepatic progression of fibrosis and steatosis by histological assessment (see below). At day minus six (−6), only animals with steatosis grade 3 and fibrosis grade ≥1 were included in the study and block randomized into treatment groups based on 1) fibrosis, 2) steatosis, and 3) body weight.

### Pre-Biopsy Procedure

Pre-biopsy: Pre-biopsies were taking three weeks prior to study start. Mice were anesthetized with isoflurane (2-3%) in 100% oxygen. A small abdominal incision was made in the midline and the left lateral lobe of the liver was exposed. A cone shaped wedge of liver tissue (approximately 50 mg) was excised from the distal portion of the lobe and fixated in 10% neutral buffered formalin (4% paraformaldehyde (PFA)) for histology. The cut surface of the liver was instantly electro coagulated using bipolar coagulation (ERBE VIO 100 electrosurgical unit). The liver was returned to the abdominal cavity, the abdominal wall was sutured and the skin was closed with staplers. For post-operative recovery mice received carprofen (5 mg/ml, 0.01 ml/10 g) administered subcutaneously (SC) on operation day and post-operation day 1 and 2. After the surgery, body weight was monitored and the animals were evaluated daily on general health and body weight.

The pre-treatment biopsy was processed as described in Histology, with regard to paraffin embedding, sectioning and Hematoxylin and Eosin (HE) staining for steatosis scoring and Sirius Red (SR) staining for fibrosis scoring. The biopsies were scored for steatosis (HE stained slides) and fibrosis (SR stained slides) according to the Kleiner score (Design and validation of a histological scoring system for nonalcoholic fatty liver disease, Kleiner et al, Hepatology 41; 2005).

### Identification

Animals were uniquely identified with implantable microchips (Pet ID Microchip, E-vet), which were implanted under light CO₂ anesthesia in all mice upon arrival. Animals were identified using the WS-1 weigh station (MBrose ApS, Faaborg, Denmark) connected to a laptop running the HM02Lab software (Ellegaard Systems, Faaborg, Denmark). The HM02Lab software matches body weight with ID and calculates dose directly based on the body weight.

### Body Weight

Body weight was monitored at baseline and daily upon dosing. Animals were offered ad libitum food and water throughout the study period, except the weight-matched group which was food restricted to lower the body weight to the same level as for animals receiving compound A or compound B.

### Compounds and Dosing

Animals were dosed once daily from day 0 up to and including day 55 and received 56 doses in total. Animals received a mean dosage of 2 nmol/kg for compound A and 3.4 nmol/kg for compound B. Animals are subjected to treatment between 8:00-9:00 AM. For SC dosing, the injection site was alternated from the left to the right side of the lower back and inguinal region, respectively.

Compounds A and B were delivered by Novo Nordisk and administered using Novo Nordisk penfills.

### Excluded Animals

During the study two animals were terminated for humane endpoints. No data from the excluded animals is included in the study. Exclusion of the animals resulted in a reduction from n=12 to n=11 in the non-alcoholic steatohepatitis (NASH) vehicle and the weight-matched vehicle groups. No animals were excluded in the healthy chow vehicle or the compound treated groups.

### Termination and Necropsy

Animals were terminated at study day 56 in a non-fasting state. Last drug dose was administered ˜20-24 hours before termination. Animals were put in anesthesia (isoflurane), the abdominal cavity was opened and cardiac blood obtained for collection of terminal plasma. Upon necropsy, whole liver was collected and weighed. The left lateral lobe was fixed in 4% PFA for histology. The medial lobe was divided into pieces and snap-frozen in liquid nitrogen for biochemical analysis and fixated in RNA later for gene expression analysis.

### Histology

Fixation, embodiment and sections: Following an over-night fixation in 4% PFA, liver biopsies were processed to paraffin in an automated Miles Scientific Tissue-TEK VIP Tissue Processor and subsequently embedded in paraffin blocks (5 biopsies per block). The blocks were cut to 3 μm sections on a Microm HM340E Microtome (Thermo Scientific). For all stains, paraffin-embedded sections were de-paraffinated in xylene and rehydrated in series of graded ethanol.

To assess hepatic steatosis sections were stained with HE: Sections were incubated for 5 min in Mayer's Hematoxylin (Cat no. S3309, Dako), washed for 5 min in running tap water and then stained for 15 sec in Eosin Y solution (Cat. No. HT110280 2.5 L, Sigma-Aldrich). Slides were hydrated, mounted with Pertex and allowed to dry before scanning. All slides were finally digitized under a 20× objective in an Aperio Scanscope AT slide scanner for histopathological scoring of steatosis.

To assess hepatic fibrosis sections were stained for SR: Another slide was stained for SR. In brief, paraffin embedded sections were de-paraffinated in xylene and rehydrated in series of graded ethanol. The slides were incubated for 10 min in Weigert's iron hematoxylin (cat no. TH107, Sigma Aldrich), washed for 5 min in running tap water, stained for 15 min in picro-sirius red (Cat no. 365548, Sigma Aldrich) and washed two times in acidified water. The remaining water was removed by vigorously shaking the slides. Thereafter the slides were hydrated in three changes of 100% ethanol, cleared in xylene and mounted with Pertex and allowed to dry before scanning as described above.

Immunohistochemistry: Sections were immune-stained for CD11b, CD45, SMA and collagen III (Col III) protein on the automated Benchmark Ultra platform (Ventana Medical Systems, Roche). In brief, sections were de-paraffinized and subjected to heat-induced antigen retrieval using CC1 pH8.4 (Roche) for 16 min. at 95° C. for CD11 b, CD45 and SMA immunostaining. For Col III immunostaining, protease-mediated antigen retrieval using Prot.1 (Roche) for 8 min. at 37° C. was applied. The monoclonal Rabbit anti-CD11 b antibody (cat. no. ab133357, Abcam) at 0.5 μg/ml, the monoclonal Rat anti-CD45 antibody (cat. no. 550539, BD Bioscience) at 5.0 μg/ml, the monoclonal Rabbit anti-SMA antibody (cat. no. ab124964, Abcam) at 4.0 μg/ml, and the polyclonal Goal anti-collagen III antibody (cat. no. 1330-01, SouthernBioTech) at 8.0 μg/ml was applied for 1 hour at 37° C. Antibody binding was detected with HRP-conjugated OmniMab anti-Rabbit antibody (Ventana) for CD11b and SMA immunostaining, and with HRP-conjugated OmniMab anti-Goat antibody (Ventana) for CD45 and Col III immunostaining. For CD45 immunostaining, Goat anti-rat IgG (cat. no. 112-005-167, Jackson ImmunoResearch) was applied as secondary linking antibody for anti-CD45 staining. Antibody binding was visualized with Purple chromogen (Ventana). Nuclei were counterstained with hematoxylin and slides were covered with PerTex. Immune-reactivity was not observed on negative control slides stained with Isotype control antibodys monoclonal Rabbit IgG [DAE1] (cat. no. 3900S, Cell Signaling Tech.), monoclonal Rat IgG2beta (cat. no. MAB0061, R&D), and polyclonal Goat IgG (cat. no. 005-000-003, Jackson ImmunoResearch) applied at the same concentration as the corresponding primary antibodies. Pre- and post-biopsies were stained in the same run to allow direct comparison.

Morphometric quantification of CD45, CD11b, SMA and Col III immunohistochemistry by Visiopharm Integrator System (VIS): In brief, all sections were scanned at 20× using the Nanozoomer 2.0 HT system (Hamamatsu, Glostrup, Denmark). Then, the scanned pictures were imported into VIS using the acquisition module. Automated image analysis was performed on scanned immunostained images with the Visiopharm Integrator System (version 4.2.2.0, Visiopharm, Horsholm, Denmark). 100% of the immunostained tissue slides were examined. The first steps in the analysis included automated tissue detection to define tissue and non-tissue boundaries, hereby defining the region of interest (ROI). Tissue detection was performed using the Visiomorph DP module, i.e. 5×5 mean on the R, G and B channel as a preprocessing step, Unsupervised K-means clustering (3 groups) as segmentation followed by a post-processing step allowing the generation of a ROI around the tissue. Inside this ROI, analysis for CD11b, CD45, SMA and Col III was performed at 20× magnification with the pre-processing step Contrast Red-Green followed by threshold analysis. A similar analysis was performed for detection of nuclei with hematoxylin. Several post-processing steps were performed to numerate nuclei (close, change by area, fill holes, separate objects, change by area, apply counting frame). The area of CD11b, CD45, SMA and Col III staining relative to the number of nuclei was calculated in each tissue section. All data were generated by doing batch analysis and data were extracted to an excel spread sheet.

### Statistics

Data are presented as the means±Standard error of mean (SEM). One-way analysis of variance (ANOVA) using Dunnett's test for multiple comparisons. For nonparametric analysis Fisher's exact test with Bonferroni correction was used. Results were considered significant at *p<0.05, **p<0.01, or ***p<0.001. All statistical analyses were performed with GraphPad Prism 7 (GraphPad Software, CA USA).

### Example 1: GLP-1 and Glucagon Receptor Binding

### Purpose

The purpose of this assay was to test the in vitro receptor binding activity (i.e. affinity) of the compounds of the invention. The receptor binding is a measure of affinity of a compound for the human GLP-1 receptor or human glucagon receptor, respectively.

### Test Compounds

The compounds used in this study are listed in Table 1. The different batches of compounds showed similar receptor affinities and potencies. Compounds were stored in a dry, dark, controlled access area and kept refrigerated at 4° C. When used in assays the compounds were dissolved in 80% DMSO/20% water prior to further dilutions in assay buffers as described in and. The final concentration of DMSO was kept at a maximum of 0.3% in all assays.

cDNAs and Plasmids

The human GLP-1 receptor cDNAs were obtained from B. Thorens (“Expression cloning of the pancreatic beta cell receptor for the gluco-incretin hormone glucagon-like peptide 1” by Thorens B. (Proc Natl Acad Sci USA 89:8641-8645,1992)). The human glucagon receptor cDNA was obtained from L. Jelinek (“The human glucagon receptor encoding gene: structure, cDNA sequence and chromosomal localization” by Lok S et al (Gene. 1994 Mar. 25; 140(2):203-9)). The cDNAs were cloned into mammalian expression vectors downstream of the CMV promoter for expression in baby hamster kidney (BHK) cells. All vector sequences were verified by sequencing, and all plasmids were purified using QIAGEN Plasmid Midi kit.

### Origin of Recombinant Cell Lines

The cell lines used for receptor binding affinity and potency assays were made by sequential, stable transfection of a BHK (wild type) cell line with first the pGL4.29[luc2P/CRE/Hygro] plasmid followed by a second transfection with a plasmid encoding either glucagon receptor or GLP-1 receptor. The cells were cultured in media containing the selection markers hygromycin and geneticin (G418). Subsequently, single clones were selected based on comparable expression levels of either a glucagon receptor or a GLP-1 receptor.

### Receptor Binding Assay

The binding assay was performed on cell membranes diluted with assay buffer (50 mM HEPES, pH 7.4 supplemented with 5 mM EGTA, 5 mM MgCl2, and 0.005% Tween 20) to achieve the final concentration of 1% or 20% plasma, respectively. The receptor binding affinities of the compounds were measured by way of their ability to displace either [¹²⁵I]-glucagon or [¹²⁵I]-GLP-1(7-36)-NH₂ using wheat germ agglutinin (WGA) scintillation proximity assay (SPA) beads (RPNQ0001) from PerkinElmer. The cells stably expressing the human glucagon or GLP-1 receptors were grown at 5% CO₂ in DMEM, 10% FCS, 0.5 mg/ml geneticin (G418) and 0.3 mg/ml hygromycin. Membrane preparations were made from cells harvested at 80% confluence, and thereafter washed and homogenized (ULTRA-TURRAX, IKA, Germany) in 20 mM HEPES and 0.1 mM EDTA buffer, pH 7.4. The protein concentration was subsequently determined, and the membranes were stored at −80° C. until use. The membrane binding assay was performed in a 96 well OptiPlate (Perkin Elmer, USA) in a total volume of 200 μl. Test compounds were dissolved in 80% DMSO and further diluted in assay buffer. Fifty μl of diluted plasma was added to the assay plate wells followed by addition of test compounds (25 μl), cell membranes (50 μl, 0.06 mg/ml), SPA beads (20 mg/ml in assay buffer) and radioligand (60,000 cpm/well, Novo Nordisk A/S). The assay plate was incubated for 2 hours at 30° C., centrifuged at 1500 rpm for 10 minutes and counted in a TopCount NXT instrument (PerkinElmer, USA).

### Calculations

The data from the TopCount instrument were transferred to GraphPad Prism software. The software averaged the values for the replicates and performed a non-linear regression. IC₅₀ values were calculated by the software and reported in nM. Data are shown in Table 1.

TABLE 1

| | Human GLP1 R | Human GCG R |
|---|---|---|
| | SPA binding | SPA binding |
| | 0% HSA | 0% HSA |
| Compound | IC₅₀ (nM) | IC₅₀ (nM) |
| Compound A | 0.3 | 1.4 |
| Compound B | 1.6 | 0.8 |

IC₅₀ values for compounds of the invention.

The results in Table 1 show that compounds of the invention are capable of binding the human GLP-1 receptor and to the human glucagon receptor in vitro.

### Example 2: Body Weight and Body Weight Changes

Purpose: The purpose of this experiment was to measure changes in total body weight during the treatment period for the different treatment groups.

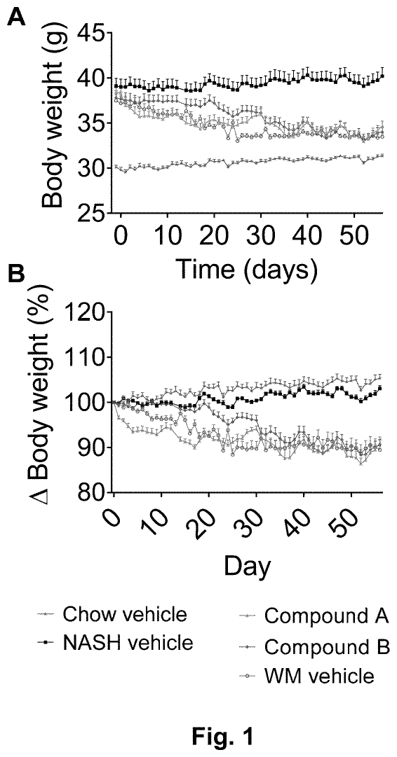

Animal experiments were conducted as described in the section “General Methods of Animal Experiments”. Animal body weight was measured as described in the subsection “Body weight”. Body weight and body weight changes are shown in FIG. 1. Data are expressed as mean±SEM. NASH groups n=11-12, Chow Vehicle group n=10.

The results in FIG. 1 show that animals treated with compounds of the invention have a decreased absolute and relative body weight compared to the NASH vehicle group. The weight-matched (WM) group has an absolute and relative body weight comparable to the compound treated groups.

### Example 3: Relative Liver Weight

Purpose: The purpose of this experiment was to measure the relative liver weight as percentage of body weight for the different treatment groups after 56 days of treatment.

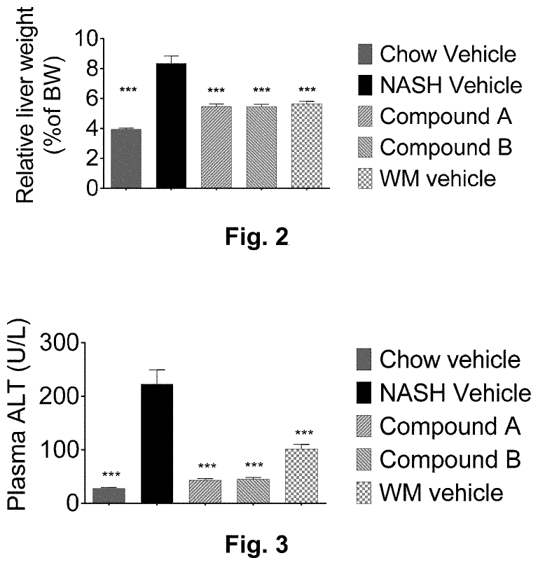

Animal experiments were conducted as described in the section “General Methods of Animal Experiments”. Animal liver weight was measured as described in the subsection “Termination and Necropsy”. Relative liver weight as percentage of body weight is shown in FIG. 2. Data are expressed as mean±SEM (NASH groups n=11-12, Chow Vehicle group n=10). One-way ANOVA with Dunnett's Multiple Comparison Test (all columns against NASH Vehicle) was used and *** indicates p<0.001 compared to NASH Vehicle.

The results in FIG. 2 show that animals treated with compounds of the invention have a significantly reduced relative liver weight as compared to the NASH vehicle group. The WM group also has a significantly reduced relative liver weight as compared to the NASH vehicle group. A reduced relative liver weight is normally observed for animals with a lower total body weight.

### Example 4: Plasma Alanine Transferase Levels

### Purpose

The purpose of this experiment was to measure plasma alanine transferase (ALT) levels. ALT is normally constrained to liver cells, but if the liver is damaged or inflamed, ALT can be released into the bloodstream. This causes plasma ALT levels to rise.

### Plasma Alanine Transferase Levels

Animal experiments were conducted as described in the section “General Methods of Animal Experiments”. Blood was collected as described in the subsection “Termination and Necropsy”. 100 μl blood was collected into Lithium-Heparin tubes. Plasma was separated and samples were stored at 4 degrees Celsius for one day prior to analysis. All parameters are measured in single determinations using autoanalyzer Cobas C-111 with commercial kit (Roche Diagnostics, Germany) according to the manufacturer's instructions. Data are shown in FIG. 3. Data are expressed as mean±SEM (NASH groups n=11-12, Chow Vehicle group n=10). One-way ANOVA with Dunnett's Multiple Comparison Test (all columns against NASH Vehicle) was used and *** indicates p<0.001 compared to NASH Vehicle.

The results in FIG. 3 show that animals treated with compounds of the invention have significantly decreased plasma ALT levels compared to the NASH vehicle group. The WM group also has significantly decreased plasma ALT levels compared to the NASH vehicle group, but the observed effect is bigger for the groups treated with either compound A or B, indicating a body weight independent effect of the compounds.

### Example 5: Histopathological Signs of Steatosis

### Purpose

The purpose of this experiment was to measure the histopathological scoring of steatosis.

### Hepatic Steatosis Assessment Using Hematoxylin and Eosin (HE)

Animal experiments were conducted as described in the section “General Methods of Animal Experiments”. Histological samples were prepared and HE stained as described in the sub-section “Histology”.

### Histopathological Scoring of Steatosis

Assessment of steatosis stage by use of clinical criteria outlined by Kleiner and colleagues (Design and validation of a histological scoring system for nonalcoholic fatty liver disease, Kleiner et al, Hepatology 41; 2005) was done on liver sections stained with HE (see description above).

| Feature | Degree | Score |
|---|---|---|
| Steatosis | <5% | 0 |
| | 5-33% | 1 |
| | >33-66% | 2 |
| | >66% | 3 |

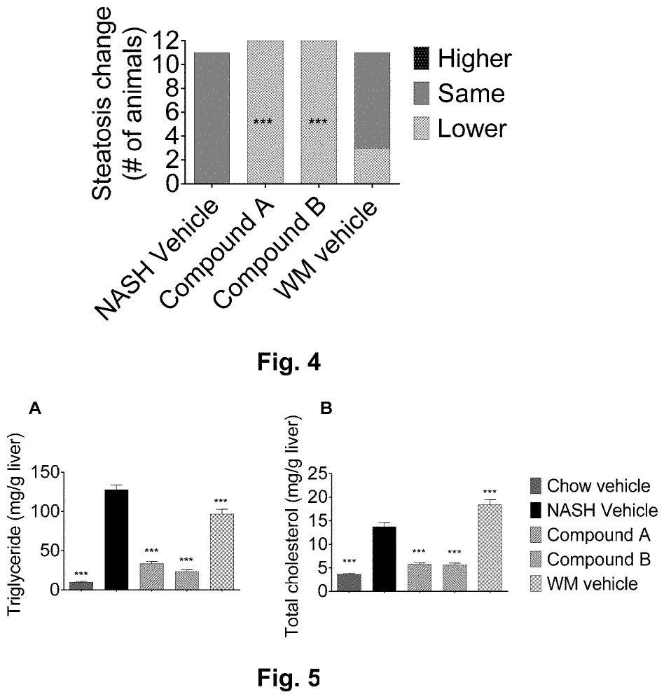

Data are shown in FIG. 4. For each group the number of animals with a higher (worsening), same, or lower (improvement) steatosis score at post-study as compared to pre-study is indicated by the height of the bar. Fisher's exact test with Bonferroni correction was used and *** indicates p<0.001.

The results in FIG. 4 show that animals treated with compounds of the invention have significantly lower (improvement) steatosis score at post-study as compared to pre-study. The same beneficial effects are not observed to the same extend for the weight-matched vehicle group, suggesting a body weight independent effect of the compounds.

### Example 6: Liver Triglycerides and Liver Cholesterol

### Purpose

The purpose of this experiment was to measure the triglycerides and cholesterol levels in the liver.

### Hepatic Terminal Triglycerides and Cholesterol Content

Animal experiments were conducted as described in the section “General Methods of Animal Experiments”. Triglycerides and total cholesterol content in liver homogenates were measured in single determinations using autoanalyzer Cobas C-111 with commercial kit (Roche Diagnostics, Germany) according to manufacturer's instructions. Data are shown in FIG. 5. Liver triglyceride levels and liver total cholesterol are given as (mg/g liver). One-way ANOVA with Dunnett's Multiple Comparison Test (all groups against NASH Vehicle) was used and *** indicates p<0.001.

The results in FIG. 5 show that animals treated with compounds of the invention have significantly reduced liver triglyceride levels and significantly reduced total liver cholesterol as compared to the NASH vehicle treatment group. The WM group has significantly reduced liver triglyceride levels, but the observed effect is bigger for the groups treated with either compound A or B. The WM group has significantly increased total liver cholesterol as compared to the NASH vehicle group. These results indicate that effects on liver triglyceride and liver total cholesterol are specific for compounds A and B.

### Example 7: Liver Inflammation

### Purpose

The purpose of this experiment was to measure markers of inflammation CD11b and CD45.

### Immunohistochemistry for CD11 b and CD45:

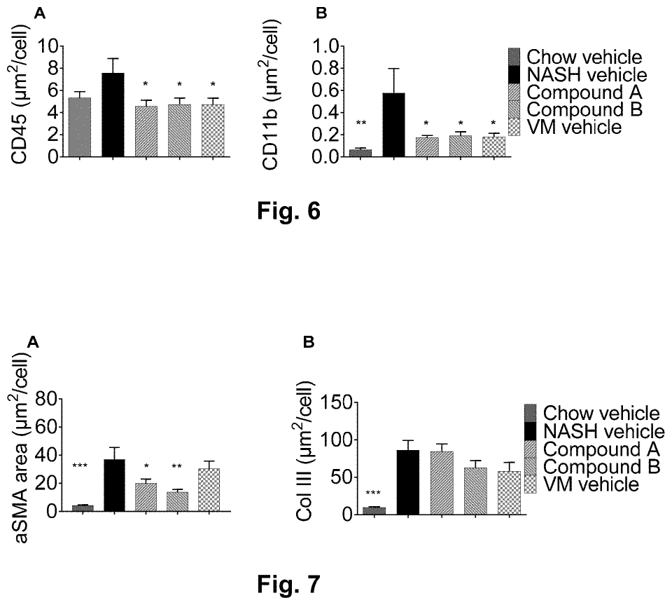

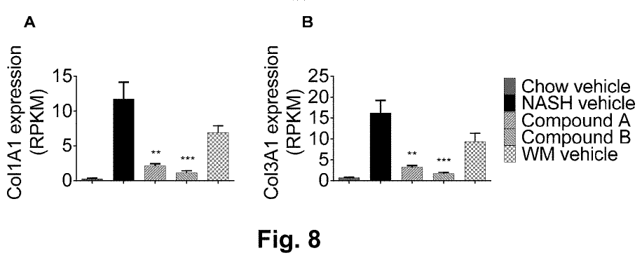

Animal experiments were conducted as described in the section “General Methods of Animal Experiments”. Histological samples were prepared, subjected to immunohistochemistry and analysed by morphometry as described in the sub-section “Histology”. Results of morphometric quantification of CD11 b and CD45 immunohistochemistry are shown in FIG. 6. Histological liver samples collected after 8 weeks (56 days) of treatment were immuno-stained for markers of inflammation. A) CD45: a pan-leucocyte marker. B) CD11 b: a marker of infiltrating monocytes, inflammatory macrophages and neutrophils. Data are presented as mean+/−SEM of stained area per number of nuclei in the sectioned biopsy (μm²/cell). n=10-12 animals/group. One-way ANOVA with Dunnett's Multiple Comparison Test (all groups compared against NASH Vehicle) was used. * indicates p<0.05 and ** indicates p<0.01.

The results in FIG. 6 show that animals treated with compounds of the invention have significantly decreased levels of the inflammatory markers CD11 b and CD45 in the liver as measured by immunohistochemistry as compared to NASH vehicle group. The WM group also has significantly decreased level of the inflammatory markers CD11 b and CD45 in the liver as measured by immunohistochemistry as compared to NASH vehicle group.

### Example 8: Liver Fibrogenesis

### Purpose

The purpose of this assay was to measure markers of fibrogenesis, Smooth Muscle Actin (SMA) and Col III.

### Immunohistochemistry for SMA and Col III:

Animal experiments were conducted as described in the section “General Methods of Animal Experiments”. Histological samples were prepared, subjected to immunohistochemistry, and analysed by morphometry as described in the sub-section “Histology”.

Data for SMA are shown in FIG. 7. Scanned slides representing stained tissue sections after 8 weeks (56 days) of treatment (post-biopsy) were analysed by digital image analysis using the VIS software. The area of SMA immune-staining in each tissue section was expressed relatively to the number of cells detected in that same tissue section. Data are presented as mean+/−SEM. One-way ANOVA with Dunnett's Multiple Comparison Test (all groups against NASH Vehicle) was used. * indicates p<0.05, ** indicates p<0.01 and *** indicates p<0.001. Data for Col III are shown in FIG. 7B. Histological liver samples collected after 8 weeks (56 days) of treatment were immuno-stained for markers of fibrogenesis. Col III: a major component of fibrogenesis. Data are presented as mean+/−SEM of stained area per number of nuclei in the sectioned biopsy (μm²/cell). n=10-12 animals/group. One-way ANOVA with Dunnett's Multiple Comparison Test (all groups against NASH Vehicle). *** indicates p<0.001.

The results in FIG. 7A show that animals treated with compounds of the invention have a significantly decreased prevalence of the fibrogenesis marker SMA in the liver as measured by immunohistochemistry as compared to NASH vehicle group. The same effects were not observed for the weight-matched control group, suggesting a body weight independent effect of the compounds.

The results in FIG. 7B show that animals treated with compounds of the invention have a decreased prevalence of the fibrogenesis marker Col III in the liver as measured by immunohistochemistry as compared to NASH vehicle group. Similar effects are observed for the weight-matched control group.

### Example 9: Liver Fibrogenesis

### Purpose

The purpose of this experiment was to measure expression levels of collagen type I, alpha 1 (Col1A1) and collagen type III, alpha 1 (Col3A1) as an indicator of liver fibrogenesis.

### RNA Sequencing

Animal experiments were conducted as described in the section “General Methods of Animal Experiments”. Lysis of tissue was performed using a MP FastPrep system. Briefly, 15-20 mg liver biopsy was homogenized and used for RNA extraction on NucleoSpin Plus RNA columns (Macherey-Nagel) as recommended by the supplier. The quantity of the RNA was assessed using a NanoDrop 2000 spectrophotometer (ThermoScientific). The purified RNA was stored at −80° C.

The NeoPrep (Illumina) was used to generate libraries for sequencing which were sequenced on a NextSeq 500 (Illumina). The sequencing data was aligned to the genome of the animal species obtained from the Ensembl database using the Spliced Transcripts Alignment to a Reference (STAR) software. For the bioinformatics analysis the quality of the data was evaluated using the standard RNA-sequencing quality control parameters. To evaluate the inter- and intra-group variability principal component analysis and hierarchical clustering was performed. To identify differentially expressed genes the R-package edgeR was used. Data are shown in FIG. 9. For each treatment the fold-change compared to vehicle is indicated and values with **p<0.01 and ***p<0.001 versus vehicle after correction for multiple testing.

The results in FIG. 9 show that animals treated with compounds of the invention have a significantly decreased expression level of Col1A1 and Col3A1 as compared to the NASH vehicle group. This indicates reduced fibrogenesis. The same effects are not observed for the weight-matched group, suggesting a body weight independent effect of the compounds.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating non-alcoholic steatohepatitis (NASH), comprising administering a therapeutically effective amount of a compound to a subject in need thereof, wherein the compound is selected from the group consisting of

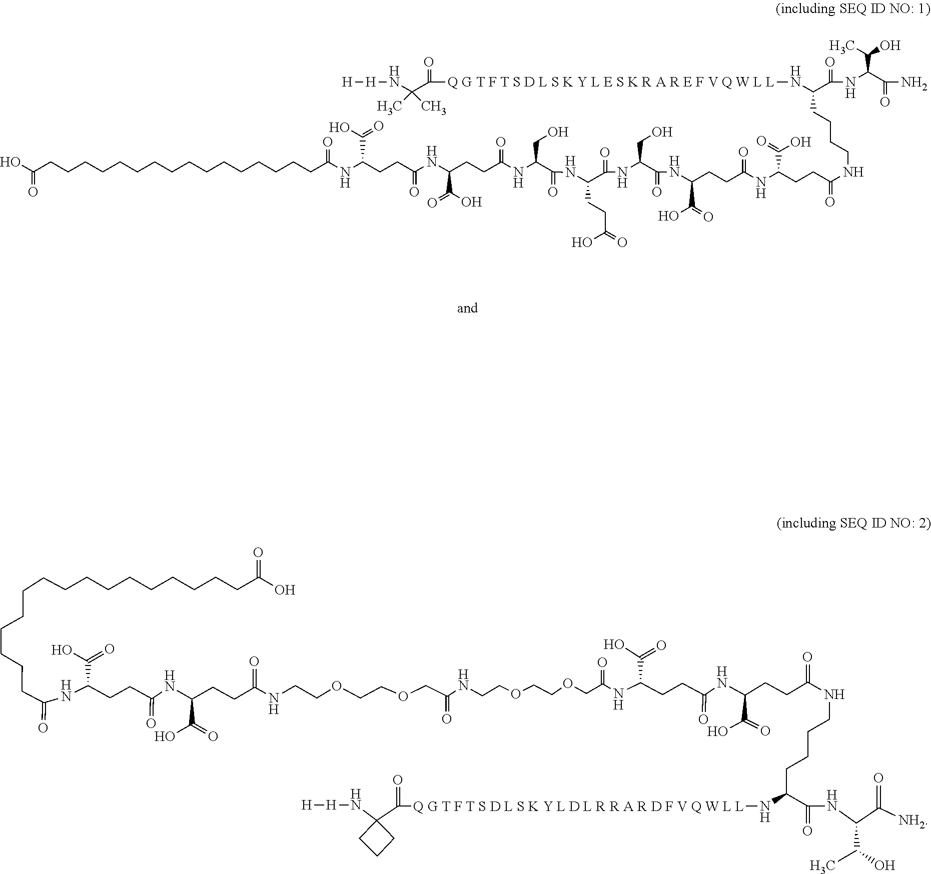

2. The method according to claim 1, wherein the compound is

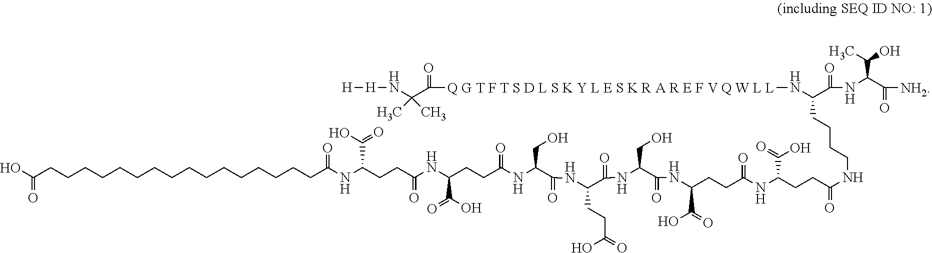

3. The method according to claim 1, wherein the compound is

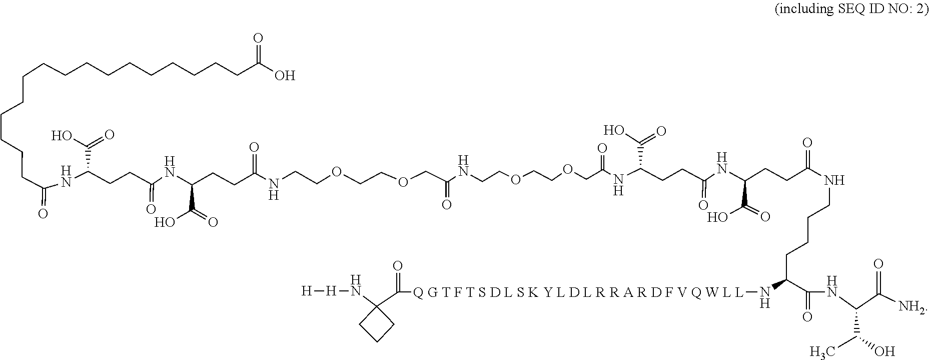

4. The method according to claim 1, wherein the compound is present in a pharmaceutical composition at a concentration in the range of 1-10 mg/ml.

5. The method according to claim 4, wherein the compound is

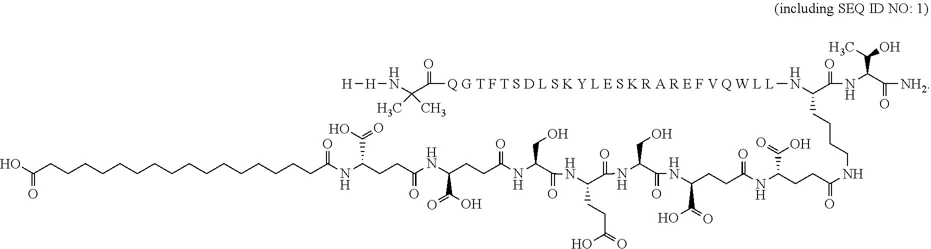

6. The compound according to claim 4, wherein the compound is

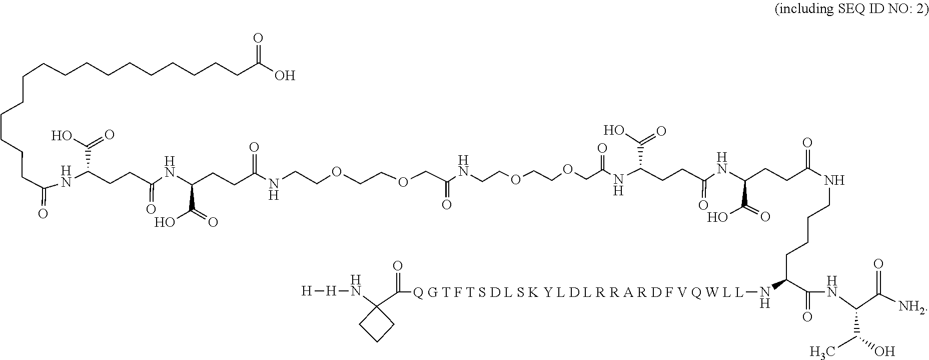

7. The method according to claim 1, wherein the compound is present in a pharmaceutical composition at an amount in the range of 0.1-10 mg.

8. The method according to claim 7, wherein the compound is

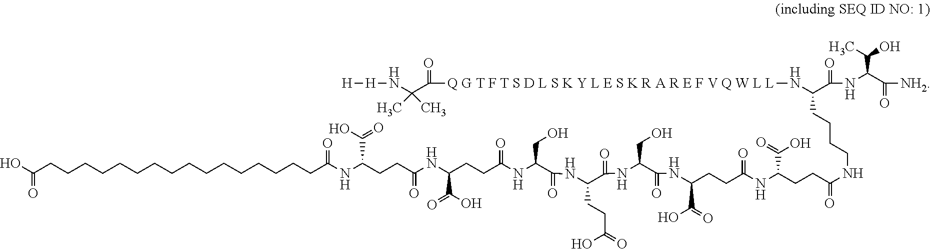

9. The method according to claim 7, wherein the compound is

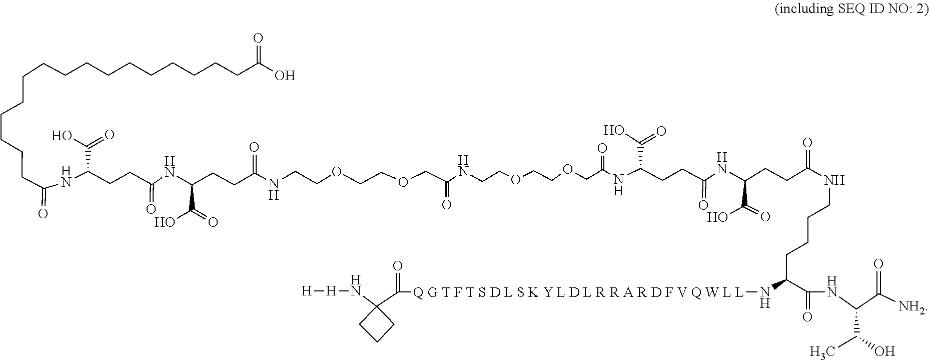

10. The method according to claim 7, wherein the compound is present in a pharmaceutical composition at a concentration of 1 mg/ml.

11. The method according to claim 10, wherein the compound is

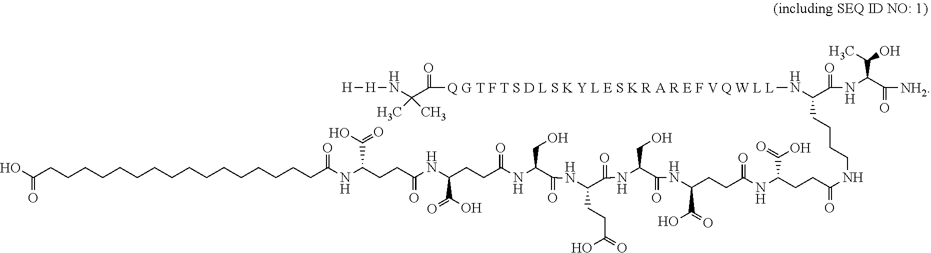

12. The method according to claim 10, wherein the compound is

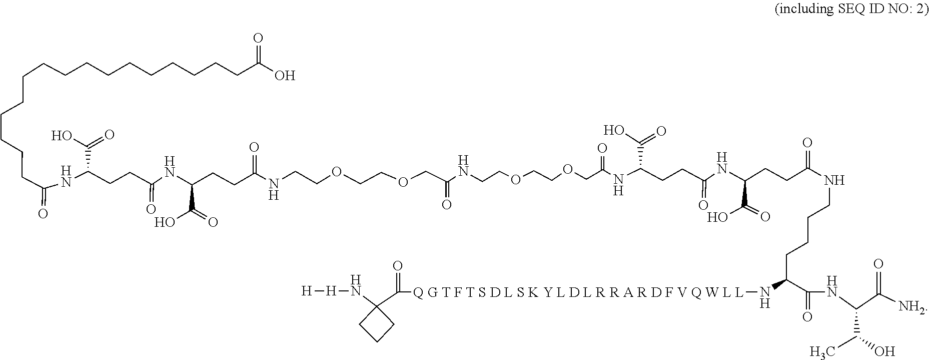

13. The method according to claim 7, wherein the compound is present in a pharmaceutical composition at a concentration of 10 mg/ml.

14. The method according to claim 13, wherein the compound is

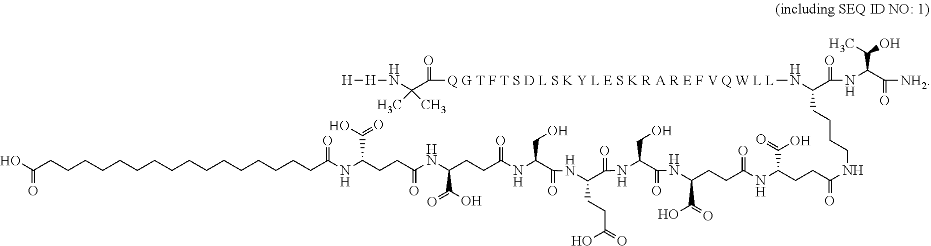

15. The method according to claim 13, wherein the compound is

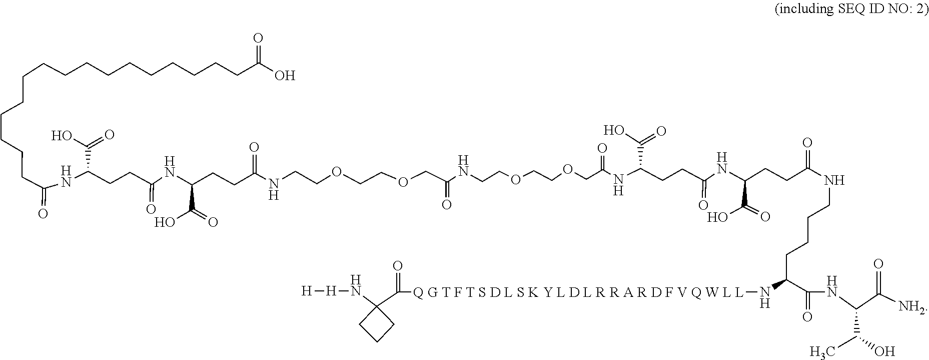

16. The method according to claim 1, wherein the subject is suffering from a condition selected from the group consisting of type I diabetes, type II diabetes, hyperglycemia, impaired glucose tolerance, overweight, and obesity.

17. The method according to claim 16, wherein the subject is suffering from type I diabetes.

18. The method according to claim 16, wherein the subject is suffering from type II diabetes.

19. The method according to claim 16, wherein the subject is suffering from overweight.

20. The method according to claim 16, wherein the subject is suffering from obesity.

21. The method according to claim 1, wherein the compound is administered subcutaneously once weekly.


SEQUENCE LISTING (2 sequences)

SEQ ID NO: 1 - protein, 29 aa, Homo Sapiens
  MISC_FEATURE (2)..(2): Xaa is Aib
HXQGTFTSDLSKYLESKRAREFVQWLLKT

SEQ ID NO: 2 - protein, 29 aa, Homo Sapiens
  MISC_FEATURE (2)..(2): Xaa is Acb
HXQGTFTSDLSKYLDLRRARDFVQWLLKT